(19) United States
(12) Patent Application Publication
(10) Pub. No.: US 2007/0244524 A1
(43) Pub. Date: Oct. 18, 2007

(54) METHOD AND DEVICE FOR PHOTOREACTIVE THERAPY AND DIAGNOSIS

(76) Inventors: Jihong Qu, Maple Grove, MN (US); Haris J. Sih, Minneapolis, MN (US)

Correspondence Address: SCHWEGMAN, LUNDBERG & WOESSNER, P.A., P.O. BOX 2938, MINNEAPOLIS, MN, 55402, US

(21) Appl. No.: 11/279,488

(22) Filed: Apr. 12, 2006

(51) Int. Cl. A61N 5/06

(52) U.S. Cl. 607/88

(57) ABSTRACT

Described are devices, systems, and methods for photoreactive operations involving the use of a photolyzable caged molecule as a photoreactive agent. The caged molecule may be a therapeutic or diagnostic agent. Also described is an implantable device configured to perform photoreactive operations in the form of photoreactive therapy and/or photoreactive diagnostic studies in either a clinical or ambulatory setting.

### FIELD OF THE INVENTION

This invention pertains to methods and devices for treating and diagnosing disease with light.

### BACKGROUND

It is known that the key of a successful pharmacological or biological (cell or gene or biological agents) therapy is the control of time and location. The maximal effect is desired only at the specific target site and at the specific moment when the treatment or diagnosis is needed. Such accurate control can not be achieved in pharmacological therapies. Indeed this is one of the biggest challenges in drug development, which is target specificity. Such control may be alternatively realized, in part, in biological therapy (cell or gene or biological agents) by using specific gene expression promoter. However specific gene expression with a promoter does not always have such an accurate temporal control with high resolution. Gene expression and alteration often do not arrive immediately after stimulation. It takes time for the effect to occur, e.g. hours to days, through gene transcription and translation.

### SUMMARY

Described herein are devices, systems, and methods for photoreactive operations involving the use of a photolyzable caged molecule as a photoreactive agent. The caged molecule may be a therapeutic or diagnostic agent. Also described herein is an implantable device configured to perform photoreactive operations in the form of photoreactive therapy and/or photoreactive diagnoses in either a clinical or ambulatory setting. Telemetry circuitry enables the device to initiate its photoreactive operations upon command or be programmed to initiate such operations according to a specified schedule. The device may also incorporate one or more sensing modalities that can be used to initiate photoreactive operations upon occurrence of a sensed event or condition. In one particular embodiment, photoreactive operational capability is incorporated into a cardiac rhythm management device that also delivers pacing and/or defibrillation therapy.

### BRIEF DESCRIPTION OF THE DRAWINGS

FIG. 1 illustrates the physical placement of an implantable device configured for photoreactive operations.

FIG. 2 illustrates the functional components of an exemplary implantable device.

FIG. 3 is a block diagram of a cardiac device with the capability of photoreactive operations.

### DETAILED DESCRIPTION

Photolabile “caged” compounds are inert precursors of biologically active molecules that can be loaded into cells and subsequently released in their active form. This is accomplished by a flash of light that triggers the “uncaging” of the bioactive molecule. The absorption of photons excites the caged molecule to an excited energy state that leads to the formation of a stable physiologically active molecule. The present disclosure deals with systems and methods for controlling, both temporally and spatially, the release and activation of therapeutic or diagnostic agents in the form of photolabile caged molecules. The caged molecule is manufactured in the way that the molecule will be locally released from its cage or chelator upon light exposure. As the term is used herein, a molecule may be a single atom or a compound. Examples of such molecules include peptides, nucleic acid fragments (e.g. RNAi), nucleotides, Ca²⁺, ATP, IP3, and cellular homeostatic probes (e.g., markers sensitive to Ca²⁺ or pH, membrane potential dyes). Examples of Ca²⁺ cages (or chelators) include DM-nitrophen, NP-EGTA, and azid-1. Nucleotides may be caged by esterfying their phosphate moieties with a photolabile nitro-phenyl ethyl (NPE) group. Other cages for blocking different chemical groups with photolyzable bonds are well-known in the art.

One example of a caged molecule is caged ATP, in which the terminal phosphate is esterfied with a blocking group (one of the derivatives of o-nitrobenzylic compounds), rendering the molecule biologically inactive until illumination. Release of caged ATP can be used, for example, to protect the heart before, during, and after a cardiac procedure (e.g. revascularization). Another example is caged IP3 (inositol 1,4,5-trisphosphate) that is manufactured the same way. IP3 can be detached from its cage intracellularly at the moment of light exposure to activate the IP3 receptor for second messenger Ca²⁺ release from the endoplasmic reticulum of muscle cells such as cardiac myocytes. Another embodiment is to use the disclosed system to treat a tumor with caged chemotherapeutic agents locally released by targeted light exposure. In another embodiment, the photolysis of a caged reporter compound (e.g. an antibody, a fluorescent marker, a secreted serum factor) can allow local monitoring and sensing of physiological function, such as the detection of cardiac excitation and contraction with a Ca²⁺ probe. Because the photoreactive sensing is only limited to the area of interest within a very brief time, global cellular toxicity that is often associated with such biomarkers is largely avoided.

A system for performing photoreactive operations as described herein may include an apparatus for delivering a photoreactive agent in the form of caged molecules and an apparatus for generating and delivering light to the caged molecules after their delivery at a target site. The caged molecules may be either therapeutic or diagnostic reagents. In the latter case, the system may also include a light sensing apparatus for collecting light from a molecule that fluoresces or otherwise released light after the molecule is released from its cage and interacts with intracellular or other components to provide diagnostic information. The light for releasing the chosen therapeutic or diagnostic reagents from the caged state can be locally delivered by optical fiber from a light source such as a laser or UV flashlamp. The spatial resolution of the effect can be as small as a few cells, whereas the temporal effect can be confined to an interval measured in microseconds to milliseconds.

In one embodiment, the photoreactive agent is delivered from an external reservoir by a catheter with a lumen or by a needle, and light is delivered internally via a fiber optic catheter in order to illuminate the photoreactive agent at the appropriate time. Light can also be collected via the fiber optic catheter when the caged molecule is a light-emitting diagnostic reagent. In another embodiment, a system for performing photoreactive operations as described above is incorporated into an implantable device that delivers the photoreactive agent to a targeted site and then delivers light for activating the agent. Such a device may include a chemical reservoir and catheter for delivering the photoreactive agent, a light source and optical fiber catheter for illuminating the photoreactive agent with light, and control circuitry for delivering the photoreactive agent and light at appropriate times. The device may also possess sensing circuitry to sense physiological variables and determine when particular events or conditions are present. The control circuitry may then initiate photoreactive operations when such events or conditions are detected.

As aforesaid, one embodiment of a system for performing photoreactive operations is an implantable device configured to deliver a caged reagent and light to internal body locations. The light generated by the device may be delivered by a lead that is attached to an implantable housing and adapted to be intravascularly or otherwise internally disposed near the target site. An example lead has a light source such as one or more light-emitting diodes (LED's) positioned at the distal end. Another embodiment utilizes an optical fiber (e.g., a catheter having an optical fiber within) that conveys light generated by a source within the housing to the target site. The light source is powered by a battery (or a rechargeable power source) within the implantable housing and emits light at a specified wavelength (e.g., one between 300 nm and 1000 nm) or combination of wavelengths. A chemical reservoir with a pump may be used to deliver the caged reagent via a catheter that can also be incorporated into a lead. The delivery of reagent and light is controlled by control circuitry within the housing which, in one embodiment, is a programmable controller that can be programmed via wireless telemetry. An exemplary device thus includes an implantable lead having a light emitting structure at its distal end and connected to an implantable housing at its proximal end, a light source for generating light that is emitted by the light emitting structure of the implantable lead, a chemical reservoir and pump for delivering a caged reagent through a catheter lead, control circuitry contained within the implantable housing operable to activate the chemical pump and the light source, and a telemetry receiver interfaced to the control circuitry to enable scheduling of photoreactive operations by wireless telemetry. Photoreactive operations may be performed acutely where the device responds to a telemetry command or chronically where the device is programmed to initiate photoreactive operations in accordance with a defined schedule or in response to sensed events. For example, the control circuitry may be programmed to deliver the caged reagent and activate the light source for a given length of time each day for a given number of days. Photoreactive operations as described above may be performed by an implantable device dedicated to that purpose or may be performed by a cardiac rhythm management device configured to also deliver cardiac therapies such as bradycardia pacing, cardioversion/defibrillation therapy, or cardiac resynchronization therapy. Implantable devices such as pacemakers and cardioverter/defibrillators are battery-powered devices which are usually implanted subcutaneously on the patient's chest and connected to electrodes by leads threaded through the vessels of the upper venous system into the heart.

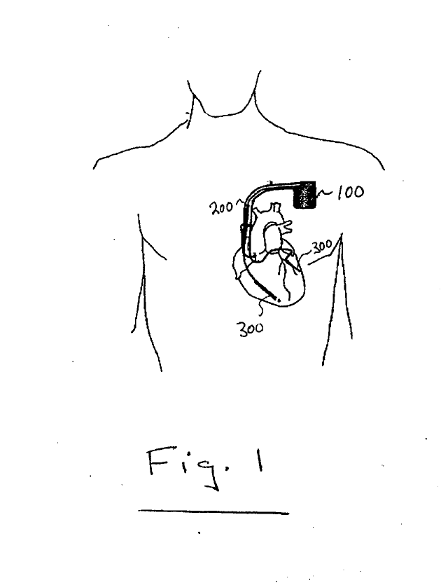

FIG. 1 shows an implantable device 100 for performing photoreactive operations that is adapted to be placed subcutaneously or submuscularly in a patient's chest with one or more leads 200 extending therefrom that are threaded intravenously into the heart. At the distal end of one of the leads 200 is a light-emitting structure 300 used to deliver light to a targeted site. As described below, a light-emitting structure 300 may be a light-emitting diode actuated and powered by a conductor within the lead 200 or may be the end of a fiber optic cable within the lead that is used to transmit light generated by the implantable device 100. Another lead 200 incorporates a catheter for delivering a caged reagent to the targeted site. The leads 200 may also include conventional leads that connect the device to electrodes used for sensing cardiac activity and for delivering electrical stimulation (i.e., either pacing pulses or defibrillation shocks) to the heart. A single lead may also be adapted for a combination of light delivery, reagent delivery, and/or cardiac sensing and stimulation.

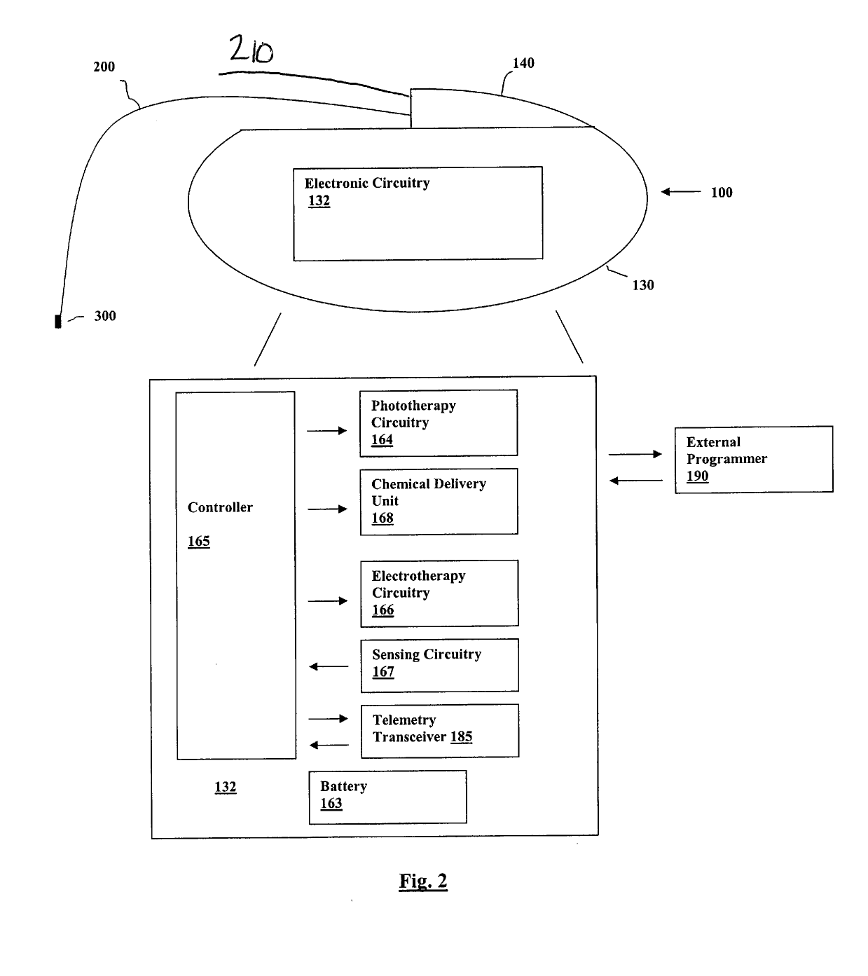

FIG. 2 illustrates an embodiment of the implantable device 100 for photoreactive operations in more detail. The device 100 includes a hermetically sealed housing 130, formed from a conductive metal, such as titanium, which may also serve as an electrode for sensing or electrical stimulation. A header 140, which may be formed of an insulating material, is mounted on housing 130 for receiving a catheter 210 used to deliver reagent out the distal end of the catheter and a lead 200 used to deliver light to an area in proximity to the light-emitting structure 300 at the end of the lead. The header may also receive leads for cardiac sensing and stimulation if the device also incorporates that functionality. Contained within the housing 130 is the electronic circuitry 132 for providing the light and reagent delivering functionality to the device as described herein and, in the case of a pacemaker or cardioverter/defibrillator, the circuitry for sensing and electrically stimulating the heart. A battery 163 provides power for the electronic circuitry 132. The electronic circuitry 132 includes a controller 165 that may be made up of discrete circuit elements but is preferably a processing element such as a microprocessor together with associated memory for program and data storage which may be programmed to perform algorithms for delivering therapy and monitoring physiological parameters. The controller 165 controls the operation of phototherapy circuitry 164, which either comprises a light-generating element (e.g., a light-emitting diode) or circuitry for actuating a light-generating element at the end of the lead 200, and a chemical delivery unit 168 comprising a reservoir and a pump for delivering the reagent via the catheter 210. A telemetry receiver or transceiver 185 capable of wirelessly communicating with an external programmer 190 is also interfaced to the controller 165. An external programmer wirelessly communicates with the device 100 and enables a clinician to issue commands to the implantable device and modify the programming of the controller. The device thus initiates photoreactive operations under programmed control as implemented in the programming of the controller 165 and may initiate such operations at programmed times and for programmed durations, in response to sensed conditions or events, or upon receiving a command to do so via telemetry.

In addition to photoreactive operational capability, the device 100 may also be configured as a pacemaker capable of delivering bradycardia and/or antitachycardia pacing, an implantable cardioverter/defibrillator, a combination pacemaker/defibrillator, a drug delivery device, or a monitoring-only device. The device 100 may be equipped for these purposes with one or more leads with electrodes for disposition in the right atrium, right ventricle, in a cardiac vein for sensing cardiac activity and/or delivering electrical stimulation to the heart, or be adapted for intra-vascular or other disposition in order to provide other types of sensing functionality. Also shown as interfaced to the controller 165 in FIG. 2 are therapy circuitry 166 for delivering electrical stimulation and sensing circuitry 167 for detecting cardiac activity as well as measuring values of other physiological parameters. For example, the sensing circuitry may include an accelerometer and/or a minute ventilation sensor for producing a signal reflective of the patient's exertion level used in rate-adaptive pacing modes. In one embodiment, a rate-responsive pacemaker is programmed to deliver and activate caged ATP when the heart's energy needs are great. At a particular rate threshold indicating a particular exertion level, ATP is released from its cage by illumination to respond the energy need for exercise.

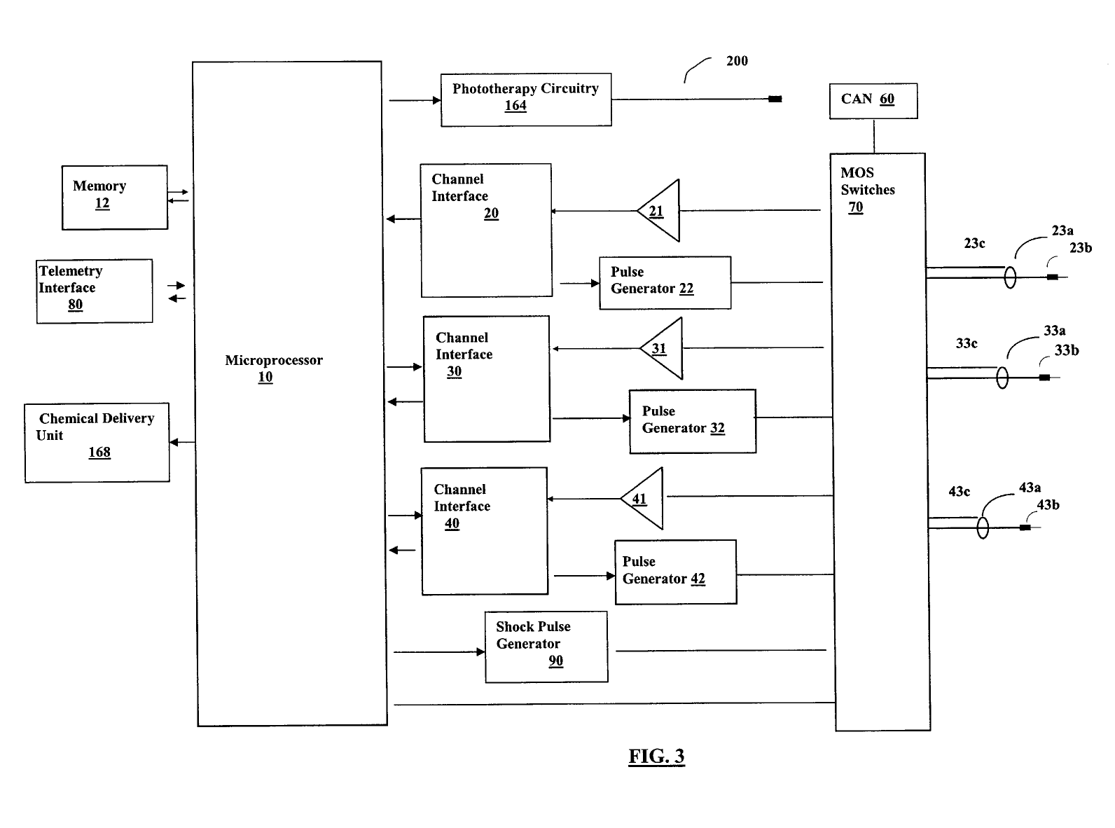

FIG. 3 is a block diagram of an implantable device with cardiac sensing, pacing, and defibrillation capability and which may be programmed to initiate photoreactive operations when certain events or conditions are detected. The controller of the device is made up of a microprocessor 10 communicating with a memory 12 via a bidirectional data bus, where the memory 12 typically comprises a ROM (read-only memory) for program storage and a RAM (random-access memory) for data storage. The controller is capable of operating the device so as to deliver a number of different therapies in response to detected cardiac activity. The controller is interfaced to chemical delivery unit 168 and to phototherapy circuitry 164, which may include an LED light source, for controlling the delivery of light through lead 200. A telemetry interface 80 is also provided for enabling the controller to communicate with an external programmer or other device via a wireless telemetry link.

The device shown in FIG. 3 has three sensing/pacing channels, where a pacing channel is made up of a pulse generator connected to an electrode while a sensing channel is made up of the sense amplifier connected to an electrode. A MOS switch matrix 70 controlled by the microprocessor is used to switch the electrodes from the input of a sense amplifier to the output of a pulse generator. The switch matrix 70 also allows the sensing and pacing channels to be configured by the controller with different combinations of the available electrodes. A shock pulse generator 90 is also interfaced to the controller for delivering defibrillation shocks between an electrode and the housing or can 60 as selected by the switch matrix. In an example configuration, a sensing/pacing channel may include ring electrode 43a (33a or 23a) and tip electrode 43b (33b or 23b) of bipolar lead 43c (33c or 23c), sense amplifier 41 (31 or 21), pulse generator 42 (32 or 22), and a channel interface 40 (30 or 20). The channel interfaces communicate bi-directionally with a port of microprocessor 10 and may include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. In the illustrated embodiment, the device is equipped with bipolar leads that include two electrodes which are used for outputting a pacing pulse and/or sensing intrinsic activity. Other embodiments may employ unipolar leads with single electrodes for sensing and pacing which are referenced to the device housing or can 60 (or another electrode) by the switch matrix 70. The channels may be configured as either atrial or ventricular channels so as to enable either biatrral or biventricular pacing.

The controller 10 controls the overall operation of the device in accordance with programmed instructions stored in memory, including controlling the delivery of paces via the pacing channels, interpreting signals received from the sensing channels, implementing timers, and delivering defibrillation shocks. The sensing circuitry of the pacemaker detects a chamber sense when an electrogram signal (i.e., a voltage sensed by an electrode representing cardiac electrical activity) generated by a particular channel exceeds a specified intrinsic detection threshold. A chamber sense may be either an atrial sense or a ventricular sense depending on whether it occurs in the atrial or ventricular sensing channel. By measuring the intervals between chamber senses, the device is able to determine an atrial or ventricular rate, and pacing algorithms used in particular pacing modes employ such senses to trigger or inhibit pacing. Measured atrial and ventricular rates are also used to detect arrhythmias such as fibrillation so that a defibrillation shock can be delivered if appropriate. In one particular embodiment, when a condition warranting delivery of a defibrillation shock is detected (e.g., ventricular fibrillation or tachycardia), the device may be programmed to deliver a caged K⁺ ion channel blocker (e.g., a Kv1.5 blocker) to the heart that is then released by illumination at the time of shocking in order to prolong the refractory period of the heart muscle cells. Delivering and activating the ion channel blocker in this manner allows for optimal anti-arrhythmic effect but least adverse effect.

The device may also be configured to detect cardiac ischemia using its sensing channels in order to deliver and activate an appropriate photoreactive agent accordingly. In order to detect whether the patient is experiencing cardiac ischemia, the controller may be programmed to analyze the recorded electrogram of an evoked response or intrinsic beat and look for a “current of injury.” A current of injury may be produced by an infarcted region that becomes permanently depolarized or by an ischemic region that remains abnormally depolarized during all or part of the cardiac cycle and results in an abnormal change in the electrical potentials measured by either a surface electrocardiogram or an intracardiac electrogram.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

What is claimed is:

1. A device, comprising:
an implantable housing;
an implantable lead having a light emitting structure at its distal end and connected to the implantable housing at its proximal end;
a light source for generating light that is emitted by the light emitting structure of the implantable lead;
a chemical delivery unit for delivering a quantity of a photoreactive agent, wherein the photoreactive agent is a caged molecule that is photolysed by light emitted from the light source;
control circuitry contained within the implantable housing operable to perform photoreactive operations by activating the chemical delivery unit and the light source.

2. The device of claim 1 further comprising a telemetry receiver interfaced to the control circuitry to enable scheduling of light emission and photoreactive agent delivery.

3. The device of claim 1 wherein the light source is contained within the implantable housing and conveys light to the light emitting structure through an optical fiber within the implantable lead.

4. The device of claim 1 wherein the light source is contained within the distal portion of the implantable lead.

5. The device of claim 1 wherein the caged molecule is calcium.

6. The device of claim 1 wherein the caged molecule is an ion-channel blocker.

7. The device of claim 1 wherein the caged molecule is a nucleotide.

8. The device of claim 7 wherein the caged molecule is ATP.

9. The device of claim 7 wherein the caged molecule is IP3.

10. The device of claim 1 further comprising:
one or more leads with electrodes for generating electrogram signals produced by cardiac activity;
sensing circuitry contained within the implantable housing for receiving the electrogram signals;
a shock generator and a lead for delivering a defibrillation shock; and,
wherein the control circuitry is programmed to cause delivery of a defibrillation shock upon detection of ventricular fibrillation from the electrogram signals and to initiate delivery of the photoreactive agent and light emission.

11. The device of claim 1 further comprising:
one or more leads with electrodes for generating electrogram signals produced by cardiac activity;
sensing circuitry contained within the implantable housing for receiving the electrogram signals; and,
wherein the control circuitry is programmed to analyze the electrogram signals and to initiate photoreactive operations if a current of injury indicative of cardiac ischemia is detected.

12. The device of claim 1 further comprising:
one or more leads with electrodes for generating electrogram signals produced by cardiac activity;
sensing circuitry contained within the implantable housing for receiving the electrogram signals;
pacing circuitry for delivering paces in accordance with a programmed mode;
exertion level measuring circuitry for measuring a physiological variable reflective of a patient's exertion level;
wherein the photoreactive agent is caged ATP; and
wherein the control circuitry is programmed to initiate delivery of the caged ATP and light emission when the measured exertion level exceeds a specified threshold value.

13. A method, comprising:
upon detection of a cardiac condition, delivering a quantity of a photoreactive agent to a target cardiac location within a patient, wherein the photoreactive agent is a caged molecule that is photolysed by light emitted from a light source; and,
delivering light to a target location that photolyzes the photoreactive agent and releases the caged molecule in order to exert a therapeutic effect.

14. The method of claim 13 further comprising delivering the photoreactive agent and light to the target location from an implantable device.

15. The method of claim 13 wherein the caged molecule is calcium.

16. The method of claim 13 wherein the caged molecule is an ion-channel blocker.

17. The method of claim 16 wherein the caged molecule is a K⁺ channel blocker.

18. The method of claim 13 wherein the caged molecule is a nucleotide.

19. The method of claim 18 wherein the caged molecule is ATP.

20. The method of claim 18 wherein the caged molecule is IP3.
